(19) United States
(12) Patent Application Publication
(10) Pub. No.: US 2009/0069585 A1
(43) Pub. Date: Mar. 12, 2009

(54) Transesterification Reacton of Triglycerides and Monohydric Alcohols

(76) Inventor: Marc E. Halpern, Williamstown, NJ (US)

Correspondence Address: Mannie Morgenstern, 15323 Weddington St., #107, Sherman Oaks, CA, 91411, US

(21) Appl. No.: 12/224,977

(22) PCT Filed: Mar. 29, 2006
(86) PCT No.: PCT/US2006/011365

§ 371 Date: Sep. 11, 2008

(51) Int. Cl. C07C 67/00; C07C 31/22

(52) U.S. Cl. 554/124; 568/869

(57) ABSTRACT

A process for producing monoesters and glycerol from triglycerides and monohydric alcohols by transesterification in the presence of phase-transfer catalysts and base initiators, produced in a batch or continuous mode. Reaction mixtures comprising triglycerides such as vegetable triglycerides, monohydric alcohols such as methanol or ethanol, base initiators such as alkali metal hydroxides or carbonates and phase-transfer catalysts such as quaternary ammonium or quaternary phosphonium salts for performing transesterification reactions. The reaction product comprises a mixture of monoesters and glycerol. The monoesters produced are useful as fuels, cleaning solvents and other industrial applications. For example, fatty acid methyl esters are produced by the transesterification reaction between fatty vegetable triglycerides and methanol in the presence of catalytic quantities of both methyl tricaprylyl ammonium chloride and sodium hydroxide at a temperature of about 70° C.

### TECHNICAL FIELD

The invention provides a process for producing monoesters and glycerol from triglycerides and monohydric alcohols by transesterification in the presence of phase-transfer catalysts and base initiators, produced in a batch or continuous mode. The invention provides reaction mixtures comprising monohydric alcohols, triglycerides, base initiators and phase-transfer catalysts for performing transesterification reactions. The reaction product comprises monoesters and glycerol.

### BACKGROUND ART

Phase-transfer catalysis is a technique for enhancing the reactivity of reactants which are soluble in one phase with reactants which are soluble in another phase, in a system in which the two phases are immiscible. Phase-transfer catalysis is used in the production of about 10 billion dollars worth of chemicals per year (as per the authoritative references: Halpern, “Phase-Transfer Catalysis” Ullmann's Encyclopedia of Industrial Chemistry, Volume A19, VCH, Weinheim, 1991, p. 293 and “Phase-Transfer Catalysis: Fundamentals, Applications and Industrial Perspectives” by Starks, Liotta and Halpern, Chapman and Hall 1994, the contents of each of which are incorporated herein by reference). The vast majority of phase-transfer catalysis applications utilize phase-transfer catalysts, which typically are an ammonium or phosphonium salt, polyethylene glycol, polyethylene glycol ether, polyethylene glycol ester or crown ether.

Triglycerides are compounds which are commercially available from natural and synthetic sources and are used as useful starting materials for various applications. Fatty triglycerides are found in vegetable oils and animal fat. Tributyrin is found in butter and used in margarine. Triacetin is used in food and cosmetic products.

Triglycerides are used in the very large scale production of bio-renewable fuels as an alternative to petroleum-based fuels. A significant amount of bio-renewable fuel, sometimes termed biodiesel, is produced by the transesterification of triglycerides with monohydric alcohols in the presence of a base initiator (often termed a “base catalyst” in prior art) to produce monoesters of fatty acids. Particularly useful monohydric alcohols used for producing monoesters of fatty esters are methanol and ethanol. Common base initiators used in these processes include sodium hydroxide and potassium hydroxide. A particularly useful raw material for such large volume commercial transesterifications is a mixture of fatty triglycerides, obtained from agricultural sources such as vegetable oil or animal fat. Vegetable oil triglycerides and animal fat triglycerides are inexpensive and are readily available from bio-renewable sources. Large commercial production facilities utilize refined fatty triglyceride oils, crude fatty triglyceride oils, waste fatty triglyceride oils, such as used frying oils, or fatty triglyceride feedstocks from various sources.

At the outset of the transesterification reaction, the reaction mixture, comprising fatty triglycerides and lower alkyl monohydric alcohols, forms two phases. At the end of the transesterification reaction, two phases are also formed but at this stage of the process the upper phase consists primarily of the monoester product and the lower phase consists primarily of glycerol and excess monohydric alcohol. Other materials may also be present in the two phases at the end of the reaction including byproducts.

Typically, in the transesterification reaction between fatty triglycerides and monohydric alcohols a base initiator is added and promotes the reaction by forming a small amount of alkoxide which is a key reactant with the fatty triglyceride. The alkoxide is generally very polar and not very soluble in the fatty triglyceride phase, especially if the alkoxide is methoxide or ethoxide. This lack of solubility constitutes a barrier for the reaction between the alkoxide and the fatty triglyceride which in turn affects throughput.

The production of monoesters of fatty acids by transesterification of fatty triglycerides with monohydric alcohols in the presence of the most common hydroxide-containing base initiators is accompanied by hydrolysis of the fatty triglycerides to form soaps. The most common hydroxide-containing base initiators are sodium hydroxide and potassium hydroxide. As published in the National Renewable Energy Laboratory Report NREL/SR-510-36244 “Biodiesel Production Technology” in July 2004 by J. Van Gerpen, B. Shanks, R. Pruszko, D. Clements and G. Knothe, “Soaps may allow emulsification that causes the separation of the glycerol and ester phases to be less sharp. Soap formation also produces water that can hydrolyze the triglycerides and contribute to the formation of more soap. Further, catalyst [i.e., base initiator] that has been converted to soap is no longer available to accelerate the reaction.” Thus, hydrolysis consumes the base initiator which reduces the rate of transesterification and can also reduce the final conversion to monoesters of fatty acids which in turn reduces throughput. Moreover, any water that is formed in the system will be in the glycerol phase at the end of the reaction and may adversely affect the quality of the glycerol which will be recovered for further use. Citing U.S. Pat. Nos. 2,383,580 and 2,383,581, the above mentioned report states “it is desirable to minimize the amount of alkali catalyst [i.e., base initiator] used because the amount of soap formed increases with increasing catalyst [i.e., base initiator]”.

In a typical process to produce a bio-renewable fuel, after the transesterification reaction, the upper phase consists primarily of methyl or ethyl esters of fatty acids and the lower phase consists primarily of glycerol and excess methanol or ethanol. The two phases are separated and treated.

After the reaction proceeds to the desired degree of completion, the rate of separation of the ester phase from the glycerol phase depends on several factors. A time limiting factor for the overall process is the time it takes for the small droplets of glycerol dispersed during the transesterification reaction to coalesce into a distinct glycerol phase once agitation is stopped at the end of the reaction. As published in the Biodiesel Production Technology report cited above “The more nearly neutral the pH, the quicker the glycerol phase will coalesce. This is one reason to minimize the total catalyst [i.e., base initiator] use.”

The Biodiesel Production Technology Report further states, “the catalyst [i.e., base initiator] tends to concentrate in the glycerol phase where it must be neutralized. The neutralization step leads to the precipitation of salts.” It is desirable to minimize the amount of base initiator used in order to facilitate the treatment and recovery of marketable glycerol as well as to minimize the wastes associated with the precipitated salts. The Biodiesel Production Technology Report states that typical base initiator loadings range from 0.3 weight % to 1.5 weight % relative to the triglyceride which is equivalent to about 6.5 mole % to about 33 mole % relative to vegetable oil.

Treatment of the ester phase typically includes washing with water. The ester washing step is used to neutralize any residual base initiator, to remove any soaps formed during the transesterification reaction, and to remove residual free glycerol and monohydric alcohol. An efficient process for the production of a suitable fuel must achieve rapid and effective separation of the phases at the end of the transesterification reaction as well as rapid separation after each water wash of the phase containing the monoester product. The presence of certain compounds reduces the rate of the separation of the phases and therefore reduces the throughput of the overall process. Such compounds which reduce the rate of separation may include fatty acids produced by hydrolysis of the fatty triglycerides as well as monoglycerides and diglycerides which may be present due to incomplete transesterification of the triglycerides. When these compounds are present in excessive amounts at the end of the reaction or during the water washes or other post reaction treatments this may result in greatly increased separation times which in turn reduces throughput.

As published in the Biodiesel Production Technology report cited above, “The presence of significant quantities of mono-, di-, and triglycerides in the final mixture can lead to the formation of an emulsion layer at the ester-glycerol interface.” The formation of such an emulsion layer generally produces one or more undesirable results including increased separation time, a net loss of product as well as the product not meeting acceptable biodiesel specifications. Thus, it is highly desirable to achieve high conversion at the end of the transesterification reaction.

### DISCLOSURE OF INVENTION

### Technical Problem

There is a need in the art for new and improved methods of producing monoesters, especially lower alkyl monoesters of fatty acids, by transesterification of triglycerides with monohydric alcohols which result in higher reactivity, more complete conversion, less use of base initiator and faster separation time to increase throughput. The invention is directed to this, as well as other, important ends.

### Technical Solution

The invention provides an improved process for performing transesterification of triglycerides with monohydric alcohols to produce monoesters. More particularly, the invention provides an improved process for performing transesterification of triglycerides with monohydric alcohols in the presence of a base initiator and a phase-transfer catalyst to produce monoesters. In one embodiment, the invention provides an improved process for performing transesterification of fatty triglycerides with monohydric alcohols in the presence of a base initiator and a phase-transfer catalyst to produce monoesters of fatty acids.

### ADVANTAGEOUS EFFECTS

In another embodiment, the invention provides reaction mixtures of phase-transfer catalysts, triglycerides, base initiators and monohydric alcohols for use in transesterification reactions that can avoid lengthy reaction time and/or lengthy separation time after the transesterification.

In another embodiment, the invention provides reaction mixtures of phase-transfer catalysts, triglycerides, base initiators and monohydric alcohols for use in transesterification reactions that utilize a reduced amount of base initiator. This can result in reduced presence of fatty acids in the reaction product which can both shorten separation time after the transesterification, as well avoid separation problems during the subsequent water washes of the monoester product. The use of a reduced amount of base initiator may also minimize the formation of salt byproducts and facilitate glycerol recovery.

In another embodiment, the invention provides reaction mixtures of phase-transfer catalysts, triglycerides, base initiators and monohydric alcohols for use in transesterification reactions at low temperatures to produce monoesters. In one embodiment, the invention provides reaction mixtures of phase-transfer catalysts, fatty triglycerides, base initiators and either methanol or ethanol for use in transesterification reactions at low temperatures or low heat histories to produce methyl esters of fatty acids or ethyl esters of fatty acids, respectively.

In another embodiment, the invention provides reaction mixtures of phase-transfer catalysts, triglycerides, base initiators and monohydric alcohols for use in transesterification reactions that achieve a more complete reaction in a short reaction time. A more complete reaction increases the likelihood of achieving a reduced amount of monoglycerides in the reaction product in a short reaction time which can avoid lengthy separation time after the transesterification. A more complete reaction will also likely help avoid separation problems during the subsequent water washes of the monoester product.

These and other aspects of the invention are described in more detail below.

### BEST MODE

The present invention provides a reaction mixture comprising at least one phase-transfer catalyst, at least one monohydric alcohol, at least one triglyceride, and at least one base initiator. In one embodiment, the triglyceride is a fatty triglyceride.

The products resulting from the transesterification process of the invention are primarily monoesters and glycerol. In one embodiment, the monoesters are fatty acid monoesters. Other components of the transesterification reaction product can include excess monohydric alcohol, fatty acids, monoglycerides, diglycerides, triglycerides, base initiator and the like.

The term “triglycerides” is intended to include any glyceryl triester or mixture of glyceryl triesters with the structure shown below:

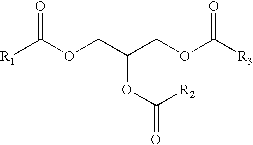

wherein R₁, R₂, and R₃ may be the same or different. Triglycerides may be derived from natural or synthetic sources, preferably from agricultural sources, more preferably from a vegetable oil and/or an animal fat. Triglycerides may be crude, refined or waste oils. Triglycerides may be a mixture of compounds or a pure compound. A mixture of triglycerides may contain lesser amounts of diglycerides, monoglycerides, glycerol and/or partially hydrolyzed glycerides. Waste triglycerides may contain other components. Preferably, triglyceryl triesters will comprise more than about 95% of a mixture containing triglycerides.

The R₁, R₂, and R₃ groups of the major triglyceride components of a mixture should contain at least one carbon atom, the majority of those R₁, R₂, and R₃ groups may preferably contain about 1 to about 40 carbon atoms, more preferably about 4 to about 30 carbon atoms, even more preferably about 12 to about 24 carbon atoms. The R₁, R₂, and R₃ groups may contain any organic functional group, preferably saturated alkyl, unsaturated or polyunsaturated groups, more preferably any functional group found naturally in any vegetable oil.

A fatty triglyceride is one in which at least one of R₁, R₂, and R₃ contain 8-40 carbon atoms; 12-30 carbon atoms; or 12-24 carbon atoms. In another embodiment, a fatty triglyceride is one in which at least two of R₁, R₂, and R₃ contain 8-40 carbon atoms; 12-30 carbon atoms; or 12-24 carbon atoms. In still another embodiment, a fatty triglyceride is one in which each of R₁, R₂, and R₃ contain 8-40 carbon atoms; 12-30 carbon atoms; or 12-24 carbon atoms.

The term “monohydric alcohol” is intended to include any aliphatic or aromatic compound containing one free hydroxyl group. For example, suitable monohydric alcohols may be selected from the following classes: saturated and unsaturated, straight and branched chain, linear aliphatics; saturated and unsaturated, cyclic aliphatics including heterocyclic aliphatics; or mononuclear and polynuclear aromatics, including heterocyclic aromatics. Preferred monohydric alcohols for use in the reaction mixture and process described herein are aliphatic alcohols, more preferably containing 1 to about 24 carbon atoms, most preferably 1 to about 12 carbon atoms. Preferred monohydric alcohols include, for example, methanol, ethanol and mixtures thereof.

A base initiator, also known in the prior art as a “basic catalyst” or a “base catalyst”, is generally used to increase the rate of reaction for the transesterification of a triglyceride with a monohydric alcohol to form esters. Base initiators may include, for example, any base or mixture of bases suitable to perform the transesterification of a triglyceride with a monohydric alcohol, including inorganic and organic bases, such as metal hydroxides, carbonates, oxides, phosphates, hydrogen phosphates, or alkoxides, preferably alkali or alkaline earth hydroxides, carbonates, oxides, or methoxides, more preferably hydroxide, oxide or carbonate salts of sodium, potassium, barium, calcium or magnesium, and most preferably hydroxide, carbonate or oxide salts of sodium, potassium or calcium. In one embodiment, the base initiator is of the formula M⁺ B⁻ wherein M is sodium, potassium, calcium, barium, or magnesium and B is hydroxide, carbonate, oxide or methoxide or mixtures thereof.

The physical form of the base initiator may be any form suitable to perform the transesterification of a triglyceride with a monohydric alcohol, including solid powder, solid granules, solid beads, or in solution.

The term “phase-transfer catalyst” is intended to include those chemical species referred to as phase-transfer catalysts in the authoritative reference “Phase-Transfer Catalysis: Fundamentals, Applications and Industrial Perspectives” by Starks, Liotta and Halpern (Chapman and Hall, 1994), the contents of which are incorporated herein by reference. Phase-transfer catalysts include quaternary ammonium salts, quaternary phosphonium salts, polyethylene glycols, polyethylene glycol ethers, polyethylene glycol esters, crown ethers, hexaalkyl guanidinium salts, complexants such as TDA-1, lariat ethers, any of the above compounds bound to polymers and mixtures of two or more thereof. Preferred phase-transfer catalysts include quaternary ammonium and phosphonium salts, polyethylene glycols and derivatives of polyethylene glycols. Quaternary ammonium or phosphonium salts used may be symmetrical or nonsymmetrical and may contain functional groups other than straight chain alkyls, such as hydroxyalkyl groups and pendant esters. Quaternary ammonium or phosphonium compounds preferably contain about 8 to about 96 carbon atoms, more preferably about 16 to about 96 carbon atoms, most preferably about 24 to about 72 carbon atoms. Quaternary ammonium or phosphonium compounds preferably contain at least three alkyl chains containing about 4 carbon atoms or more each, more preferably contain at least three alkyl chains containing about 8 to about 18 carbon atoms each. Quaternary ammonium or phosphonium compounds containing at least three alkyl chains containing about 4 carbon atoms or more each, preferably about 8 carbon atoms or more each, minimize the formation of undesirable stable emulsions at the end of the transesterification reaction. The anion of the quaternary onium salt may be any anion, preferably an inorganic anion, more preferably chloride, bromide, acetate, fluoride, nitrate, hydroxide, iodide, hydrogen sulfate, sulfate, methylsulfate and carbonate. When the anion contains an acidic hydrogen, such as hydrogen sulfate or hydrogen phosphate, additional base initiator should be added to neutralize the acidic hydrogen.

When the triglyceride is a fatty triglyceride, quaternary ammonium or phosphonium salts preferably contain about 20 to about 96 carbon atoms, more preferably about 24 carbon atoms to about 96 carbon atoms, most preferably about 24 carbon atoms to about 72 carbon atoms.

Polyethylene glycol and derivatives are of the form R—O—[(CHY)—CH₂O]ₙ—R′, wherein R is a hydrogen atom or an alkyl containing about 1 to about 24 carbons or ester containing about 1 to about 24 carbon atoms, R′ is a hydrogen atom or an alkyl containing about 1 to about 24 carbon atoms or an ester containing about 1 to about 24 carbons, Y is a hydrogen atom or methyl. Preferably Y is a hydrogen atom. In the above formula, n is about 2 to about 150, preferably about 4 to about 35. R and R′ may or may not be the same. Polyethylene glycol and derivatives preferably do not form stable emulsions at the end of the transesterification reaction.

In another embodiment, the phase transfer catalyst is a compound of the formula RₐR_{b}R_{c}R_{d}A⁺ X⁻; a compound of the formula R″—(OCH₂CH₂)ₙ—OR′″; or a mixture thereof; wherein A is nitrogen or phosphorous, Rₐ, R_{b}, R_{c} and R_{d} are individually straight chain C₁-C₂₄ alkyl groups, X is chloride, bromide, acetate, fluoride, nitrate, hydroxide, iodide, hydrogen sulfate, sulfate, methylsulfate or carbonate, R″ and R′″ individually are hydrogen, an alkyl group of about 1 to about 24 carbon atoms, or an esterified carboxylic acid, and n is about 2 to about 150.

In other embodiments, the phase-transfer catalyst is a compound of the formula RₐR_{b}R_{c}R_{d}A⁺ X⁻ or a mixture thereof; wherein A is nitrogen, Rₐ is a straight chain C₁-C₂₄ alkyl group and R_{b}, R_{c}, and R_{d} are individually straight chain C₈-C₂₄ alkyl groups such as salts of methyl tricaprylyl ammonium, methyl trioctyl ammonium, methyl tridecyl ammonium, methyl tridodecyl ammonium, tetracaprylyl ammonium, tetraoctyl ammonium, tetradecyl ammonium or tetradodecyl ammonium. In still other embodiments, the phase transfer catalyst is a polyethylene glycol of the formula R″—(OCH₂CH₂)ₙ—OR′″ wherein R″ and R′″ individually are hydrogen, an alkyl group of about 1 to about 8 carbon atoms, and n is about 4 to about 50.

The transesterification reaction may be performed at any temperature suitable to obtain substantial reactivity. The transesterification reaction may be performed at a temperature of about 0° C. to about 150° C., preferably at about room temperature to 80° C., more preferably at about room temperature to about 75° C. When the transesterification is performed at a temperature above the boiling point of the any of the components, the transesterification may be performed at a pressure above atmospheric pressure.

The transesterification reaction can proceed about 10 seconds to about 10 hours or more, depending on the temperature, amount and identity of the phase-transfer catalyst, the identity and amount of the base initiator and other factors. Preferably, the transesterification reaction is performed about 1 minute to about 1 hour.

The transesterification reaction can be performed in a batch or continuous mode. If performed in a continuous mode, the transesterification reaction is preferably performed in a plug flow reactor or in a continuous stirred tank reactor or in a series of continuous stirred tank reactors. If performed in a batch mode, the transesterification reaction may be performed in a series of batch reactors.

The transesterification may be performed using a ratio of monohydric alcohol to triglyceride which is suitable to obtain the desired product and will depend on the identities of the monohydric alcohol and the triglyceride. Such ratios are routinely determined by one of ordinary skill in the art. The molar ratio of monohydric alcohol to triglyceride may be about 3 to about 20, preferably about 3.5 to about 10, more preferably about 4 to about 8.

The transesterification may be performed in the presence of any quantity of phase-transfer catalyst suitable to obtain substantial reactivity. The quantity of phase-transfer catalyst expressed in terms of molar ratio relative to triglyceride may be in the range of about 0.001 mole % to about 10 mole %, preferably about 0.1 mole % to about 5 mole %, more preferably 0.25 mole % to about 2 mole %.

The transesterification may be performed in the presence of any quantity of base initiator suitable to obtain substantial reactivity and substantial conversion and preferably avoid substantial hydrolysis of the triglyceride. The quantity of base initiator relative to triglyceride may be in the range of about 0.001 mole % to about 50 mole %, preferably about 0.1 mole % to about 20 mole %, more preferably about 0.5 mole % to about 10 mole %. In another embodiment, free acids or free fatty acids may be present in the reaction mixture prior to the addition of the base initiator. Typically this is determined by titrating the triglyceride for acid content. When acid is determined to be present in the triglyceride, the acids need to be neutralized by an equivalent quantity of base before adding the quantity of base initiator cited in this paragraph.

In one embodiment, the reaction mixture is substantially solvent free, where “substantially solvent free” means that the reaction mixture contains solvent(s) in an amount of 25% by weight or less; 20% by weight or less; 15% by weight or less; 10% by weight or less; 5% by weight or less; or 1% by weight or less. In another embodiment, the reaction mixture does not contain any solvent.

### Mode for Invention

### EXAMPLES

The invention is further illustrated by the following non-limiting examples.

### Example 1

The reaction vessel was a 250 mL 3-necked round bottom flask equipped with an agitator consisting of a shaft with a single half moon teflon blade of dimensions 45 mm wide and 20 mm high, a thermometer set to be immersed below the liquid level and a sample port. The flask was immersed in a temperature controlled water bath. The agitator was controlled by a Heidolph RZR-1 variable speed motor and the stirring speed was measured by a VWR photo tachometer.

To the reaction vessel were added 0.44 grams methyl tricaprylyl ammonium chloride (Aliquat® 336 from Cognis Corporation; 1.0 mmole assuming a molecular weight of 432 g/mole) as the phase-transfer catalyst and 90.58 grams soybean oil (Crisco® brand lot # 5332420; 104 mmoles assuming a molecular weight of 872 grams/mole). The agitator was started at 300+/−2 rpm and the phase-transfer catalyst dissolved rapidly. A fresh solution was prepared by dissolving 0.158 grams sodium hydroxide 3 mm flakes (97% min from Aldrich Chemical; 4.0 mmoles) as the base initiator in 15.56 grams methanol (99.9%+HPLC grade from J. T. Baker; 486 mmoles). The methanol solution was added to the soybean oil solution at 24° C. The reaction temperature was 22-24° C. Weighed samples were taken from the agitated reaction mixture at 10 min, 20 min, 30 min, 40 min, 60 min, 80 min and 120 min. Each sample was diluted in a weighed amount of heptane, decane, tetradecane (internal standard for gas chromatography) and water. Five drops of the upper layer were treated with 10 drops of pyridine and 5 drops of BSTFA (bis[trimethylsilyl]trifluoroacetamide; refrigerated 99%+derivatization grade from Aldrich) and heated to 65° C. for 20 min. Analysis was performed on a HP-1 capillary column 30 m long, 0.32 mm ID with a 0.25 μm film thickness in an HP 6850 gas chromatgraph equipped with a thermal conductivity detector. The carrier gas was helium with a flow rate of 2.00 mL/min through the column split at a 50:1 ratio. The injector and detector were at 250° C. The temperature program was from 150° C. to 250° C. increased at 20° C./min and held at 250° C. for 10 min. Conversion was determined by comparing the GC results of each sample to a standard of 99% methyl soyate prepared from the same batch of soybean oil using 8 moles of methanol per mole of soybean oil and washed three times and verified by methyl stearate analysis using a 4-point calibration curve of methyl stearate to tetradecane with an R² value of 0.999. The results are shown in Table 1 below.

TABLE 1
Conversion of soybean oil to soybean methyl ester in the transesterification
reaction performed at 22-24° C. using 0.17 wt % NaOH (3.8 mole %) relative
to soybean oil, with and without 1 mole % phase-transfer catalyst and a 5:1 volume
ratio of soybean oil to methanol (4.7 equiv methanol to soybean oil)

| | Cyphos ® 3653 | Aliquat ® 336 | no PTC | Bu₄NOAc | Bu₄NHSO₄ |
|---|---|---|---|---|---|
| Time (min) | Example 3 | Example 1 | Example 2 | Example 4 | Example 5 |
| 10 | 26% | 18% | 11% | | |
| 20 | 57% | 41% | 25% | 28% | 14% |
| 30 | 64% | 59% | 38% | 37% | 22% |
| 40 | 73% | 63% | 45% | 43% | 25% |
| 60 | 83% | 77% | 51% | 54% | 34% |
| 80 | 94% | 83% | 59% | 58% | 38% |
| 120 | 98% | 94% | 66% | 64% | 44% |

### Comparative Example 2

The reaction vessel described in Example 1 was used and the procedure described in Example 1 was performed using no phase-transfer catalyst, 90.90 grams of soybean oil (104 mmoles assuming a molecular weight of 872 grams/mole) and a fresh solution of 0.158 grams of NaOH flakes (4.0 mmoles) as the base initiator in 15.37 grams of methanol (480 mmoles). The results are shown in Table 1 above and clearly demonstrate that the reaction with Aliquat® 336 achieved significantly higher conversion at significantly shorter time than the reaction without the phase-transfer catalyst.

### Example 3

The reaction vessel described in Example 1 was used and the procedure described in Example 1 was performed using 0.55 grams CYPHOS® 3653 (trademark of Cytec; trihexyl tetradecyl phosphonium chloride 99%, 1.0 mmole) as the phase-transfer catalyst, 90.69 grams of soybean oil (104 mmoles assuming a molecular weight of 872 grams/mole) and a fresh solution of 0.163 grams of NaOH flakes (4.1 mmoles) as the base initiator in 15.33 grams of methanol (479 mmoles). The results are shown in Table 1 above and clearly demonstrate that the reaction with CYPHOS® 3653 achieved significantly higher conversion at significantly shorter time than the reaction without the phase-transfer catalyst.

### Example 4

The reaction vessel described in Example 1 was used and the procedure described in Example 1 was performed using 0.25 grams tetrabutyl ammonium acetate (98% from Sachem, 1.0 mmole; the tetrabutyl ammonium acetate did not appear to dissolve in the soybean oil) as the phase-transfer catalyst, 90.73 grams of soybean oil (104 mmoles assuming a molecular weight of 872 grams/mole) and a fresh solution of 0.156 grams of NaOH flakes (3.9 mmoles) as the base initiator in 15.42 grams of methanol (482 mmoles). The results are shown in Table 1 above. This Example demonstrates that a quaternary ammonium phase-transfer catalyst with 16 carbon atoms is not lipophilic enough to enhance reactivity for transesterification of vegetable fatty triglycerides

### Example 5

The reaction vessel described in Example 1 was used and the procedure described in Example 1 was performed using 0.34 grams tetrabutyl ammonium acetate (99% from Dishman, 1.0 mmole; the tetrabutyl ammonium hydrogen sulfate did not appear to dissolve in the soybean oil) as the phase-transfer catalyst, 90.24 grams of soybean oil (103 mmoles assuming a molecular weight of 872 grams/mole) and a fresh solution of 0.158 grams of NaOH flakes (4.0 mmoles) as the base initiator in 15.33 grams of methanol (479 mmoles). The results are shown in Table 1 above. This Example demonstrates that a quaternary ammonium phase-transfer catalyst with an acidic hydrogen consumes a portion of the base initiator which results in a reduced rate of transesterification.

### Example 6

The reaction vessel described in Example 1 was used and the procedure described in Example 1 was performed at a temperature of 52-54° C. using 0.40 grams Aliquat® 336 (0.93 mmoles) as the phase-transfer catalyst and 90.02 grams soybean oil (103 mmoles assuming a molecular weight of 872 grams/mole). To the reaction vessel were added 1.19 grams granular potassium carbonate (from Aldrich Chemical; 8.6 mmoles) as the base initiator and 20.0 mL methanol (99.9%+HPLC grade from J. T. Baker. Sodium hydroxide was not used in this example. Samples were taken at 20 min, 40 min and 60 min. The results reported in Table 2 below show the % monoglyceride remaining compared to the total of methyl ester and monoglyceride.

The reaction was stopped after 72 min, transferred to a separatory funnel and the lower phase was drained. The upper phase was vigorously shaken with 30 mL of tap water until it had the appearance of an emulsion. Within 6 minutes, the lower aqueous phase was observed to be totally clear and fully separated from the upper phase. The lower aqueous phase was drained after a total of 13 min. The upper phase was treated a second time with 30 mL of tap water and the aqueous phase was observed to be totally clear and fully separated from the upper phase within 7 min. The lower aqueous phase was drained after a total of 10 min. The upper phase was treated a third time with 30 mL of tap water and the aqueous phase was observed to be totally clear and fully separated from the upper phase within 7 min.

### Comparative Example 7

The reaction vessel described in Example 1 was used and the procedure described in Example 3 was performed at a temperature of 52-54° C. using no phase-transfer catalyst and 90.11 grams soybean oil (103 mmoles assuming a molecular weight of 872 grams/mole). To the reaction vessel were added 1.22 grams granular potassium carbonate (from Aldrich Chemical; 8.8 mmole) as the base initiator and 20.0 mL methanol. Sodium hydroxide was not used in this example. The results are shown in Table 2 below.

The reaction was stopped after 72 min, transferred to a separatory funnel and the lower phase was drained. The upper phase was vigorously shaken with 30 mL of tap water until it had the appearance of an emulsion. The aqueous phase was observed to still be a cloudy white milky emulsion after 60 min.

The results shown in Table 2 clearly demonstrate that the reaction with Aliquat® 336 achieved lower amounts of monoglyceride in the reaction mixture at a shorter time than the reaction without the phase-transfer catalyst. The results of the water washes at the end of the reaction clearly demonstrate that the reaction with Aliquat® 336 provides better separation during the post reaction treatment.

TABLE 2
Amount of monoglyceride remaining in the reaction mixture relative
to methyl ester in the reaction performed at 52-54° C. using 1.3
wt % K₂CO₃ relative to soybean oil, with and without Aliquat ® 336
and a 5:1 volume ratio of soybean oil to methanol

| | | With 0.44 wt % Aliquat ® 336 |
|---|---|---|
| Time | No phase-transfer catalyst | relative to soybean oil |
| 20 min | 9% | 7% |
| 40 min | 5% | 3% |
| 60 min | 4% | 2% |

### Example 8

The reaction vessel described in Example 1 was used. To the reaction vessel were added 0.43 grams Aliquat® 336 (1.0 mmole) as the phase-transfer catalyst, 91.19 grams of soybean oil (105 mmoles assuming a molecular weight of 872 grams/mole) and 3.00 grams of tetradecane as internal standard. The agitator was started at 300+/−2 rpm and the phase-transfer catalyst, internal standard and oil dissolved readily to form a single phase. To the flask were then added 15.44 grams of methanol (483 mmoles). The flask was immersed in a hot water bath, fitted with a reflux condenser. Samples for GC analysis were taken during the heating period and it was verified that no conversion to monoesters occurred. When the contents of the flask reached a stable temperature of 65° C., 48 milligrams of NaOH (20-40 mesh beads from Aldrich; 1.2 mmole) as base initiator were added to the reaction mixture. The reaction temperature reached 70° C. within 6 minutes and remained at 70° C. for the duration of the reaction which was 60 minutes total.

Samples were taken from the agitated reaction mixture at 3 min, 6 min, 15 min, 40 min and 60 min. The results are shown in Table 3 below.

At the end of the reaction, the contents of the flask were transferred to a separatory funnel and the lower phase was drained. The upper phase was vigorously shaken with 30 mL of tap water until it had the appearance of an emulsion. Within 10 minutes, the lower aqueous phase was observed to be fully separated from the upper phase and was nearly totally clear.

### Comparative Example 9

The reaction vessel described in Example 1 was used and the procedure described in Example 8 was performed using no phase-transfer catalyst, 90.68 grams of soybean oil (104 mmoles assuming a molecular weight of 872 grams/mole), 3.00 grams of decane as internal standard and 15.41 grams of methanol (482 mmoles). When the contents of the flask reached a stable temperature of 65° C., 45 milligrams of NaOH (20-40 mesh beads from Aldrich; 1.1 mmole) as base initiator were added to the reaction mixture. The reaction temperature reached 70° C. within 6 minutes and remained at 70° C. for the duration of the reaction which was 60 minutes total. Samples were taken from the agitated reaction mixture at 3 min, 6 min, 15 min, 40 min and 60 min. The results are shown in Table 3 below and clearly demonstrate that the reaction using a reduced amount of base initiator with Aliquat® 336 achieved significantly higher conversion at significantly shorter time than the reaction without the phase-transfer catalyst.

At the end of the reaction, the contents of the flask were transferred to a separatory funnel and the lower phase was drained. The upper phase was vigorously shaken with 30 mL of tap water until it had the appearance of an emulsion. After 30 min, the lower aqueous phase was observed to be a totally opaque white milky emulsion. Comparison of the observations of the separation of the aqueous phase of the water washes in Examples 8 and 9 clearly demonstrate that the use of Aliquat 336 with the lower amount of base initiator results in better separation during the water wash treatment.

TABLE 3
Conversion of soybean oil to soybean methyl ester in the transester-
ification reaction performed at 65-70° C. using 0.05 wt % NaOH
(1.1 mole %) relative to soybean oil, with and without 1 mole %
phase-transfer catalyst and a 5:1 volume ratio of soybean oil to
methanol (4.7 equiv methanol to soybean oil).

| time (min) | With Aliquat 336 | no phase-transfer catalyst |
|---|---|---|
| 3 | 32% | 21% |
| 6 | 58% | 48% |
| 15 | 66% | 53% |
| 40 | 78% | 60% |
| 60 | 82% | 62% |

It will be appreciated by one of ordinary skill in the art that changes could be made to the embodiments described above without departing from the broad inventive concept thereof. Other triglycerides, monohydric alcohols, base initiators and phase-transfer catalysts other than those specifically mentioned in the above examples may be used to create other embodiments of the present invention. It is understood, therefore, that this invention is not limited to the particular embodiments disclosed, but is intended to cover modifications within the spirit and scope of the present invention as defined by the appended claims.

### INDUSTRIAL APPLICABILITY

This invention improves the performance of industrial manufacturing facilities that use transesterification of triglycerides with monohydric alcohols to produce monoesters of significant commercial value. Monoesters are used in many industrial applications including use as solvents and fuels. In particular, monoesters of fatty acid are used in large quantity as industrial cleaning solvents and bio-renewable fuels and are produced by transesterification of vegetable and animal triglycerides, commonly using methanol or ethanol as the monohydric alcohol. Large scale biodiesel plants often use vegetable oils that are readily available in crude and/or refined form including but not limited to soybean oil and canola oil in North America, rapeseed oil in Europe and palm oil and jatropha oil in Asia or from waste sources including but not limited to waste vegetable oil, yellow grease or trap grease.

What is claimed is:

1. A reaction mixture for synthesizing monoesters comprising at least one phase-transfer catalyst, at least one triglyceride, at least one base initiator, and at least one monohydric alcohol.

2. The reaction mixture of claim 1, wherein a phase-transfer catalyst is a quaternary ammonium salt, a quaternary phosphonium salt, a polyethylene glycol, a polyethylene glycol ether, a polyethylene glycol ester, a crown ether, a hexaalkyl guanidinium salt, TDA-1, a lariat ether, any of the above compounds bound to a polymer, a derivative thereof or a mixture of at least two thereof.

3. The reaction mixture of claim 1, wherein a phase transfer catalyst is a quaternary ammonium salt or quaternary phosphonium salt containing 8 to 96 carbon atoms, of the formula RₐR_{b}R_{c}R_{d}A^{+X}⁻ wherein A is nitrogen or phosphorous, Rₐ, R_{b}, R_{c}, and R_{d} are individually straight chain C₁-C₂₄ alkyl groups, X is chloride, bromide, acetate, fluoride, nitrate, hydroxide, iodide, hydrogen sulfate, sulfate, methylsulfate or carbonate.

4. The reaction mixture of claim 1, wherein a phase-transfer catalyst is a compound of the formula RₐR_{b}R_{c}R_{d}A⁺X⁻ or a mixture thereof wherein A is nitrogen, Rₐ is a straight chain C₁-C₂₄ alkyl group and R_{b}, R_{c} and R_{d} are individually straight chain C₈-C₂₄ alkyl groups.

5. The reaction mixture of claim 1, wherein a triglyceride is derived from crude vegetable oil, refined vegetable oil, waste vegetable oil and/or animal fat.

6. The reaction mixture of claim 1, wherein a base initiator is of the formula M⁺ B⁻ wherein M is an alkali metal or an alkaline earth metal and B is hydroxide, carbonate, oxide, alkoxide, phosphate, hydrogen phosphate or mixtures thereof.

7. The reaction mixture of claim 1, wherein a monohydric alcohol is an aliphatic compound having one free hydroxyl group and 1 carbon atom to 24 carbon atoms or a mixture thereof.

8. The reaction mixture of claim 1, wherein a monohydric alcohol is methanol, ethanol or a mixture thereof.

9. The reaction mixture of claim 1, wherein the molar ratio of monohydric alcohol to triglyceride is in the range of 3:1 to 20:1.

10. The reaction mixture of claim 1, wherein the molar ratio of phase-transfer catalyst to triglyceride is in the range of 0.1 mole % to 5 mole %.

11. The reaction mixture of claim 1, wherein the molar ratio of base initiator to triglyceride is in the range of 0.1 mole % to 20 mole %.

12. A method for synthesizing monoesters comprising mixing at least one phase-transfer catalyst, at least one triglyceride, at least one base initiator, and at least one monohydric alcohol.

13. The method of claim 12, further comprising heating the resulting mixture to a temperature in the range of 25° C. to 150° C.

14. The method of claim 12, wherein a phase-transfer catalyst is a quaternary ammonium salt, a quaternary phosphonium salt, a polyethylene glycol, a polyethylene glycol ether, a polyethylene glycol ester, a crown ether, a hexaalkyl guanidinium salt, TDA-1, a lariat ether, any of the above compounds bound to a polymer, a derivative thereof or a mixture of at least two thereof.

15. The method of claim 12, wherein a phase transfer catalyst is a quaternary ammonium salt or quaternary phosphonium salt containing 8 to 96 carbon atoms, of the formula RₐR_{b}R_{c}R_{d}A⁺ X⁻ wherein A is nitrogen or phosphorous, Rₐ, R_{b}, R_{c}, and R_{d} are individually straight chain C₁-C₂₄ alkyl groups, X is chloride, bromide, acetate, fluoride, nitrate, hydroxide, iodide, hydrogen sulfate, sulfate, methylsulfate or carbonate.

16. The method of claim 12, wherein a phase-transfer catalyst is a compound of the formula RₐR_{b}R_{c}R_{d}A⁺ X⁻ or a mixture thereof wherein A is nitrogen, Rₐ is a straight chain C₁-C₂₄ alkyl group and R_{b}, R_{c}, and R_{d} are individually straight chain C₈-C₂₄ alkyl groups.

17. The method of claim 12, wherein a triglyceride is derived from crude vegetable oil, refined vegetable oil, waste vegetable oil and/or animal fat.

18. The method of claim 12, wherein a base initiator is of the formula M⁺ B⁻ wherein M is an alkali metal or an alkaline earth metal and B is hydroxide, carbonate, oxide, alkoxide, phosphate, hydrogen phosphate or mixtures thereof.

19. The method of claim 12, wherein a monohydric alcohol is an aliphatic compound having one free hydroxyl group and 1 carbon atom to 24 carbon atoms or a mixture thereof.

20. The method of claim 12, wherein a monohydric alcohol is methanol, ethanol or a mixture thereof.

21. The method of claim 12, wherein the molar ratio of monohydric alcohol to triglyceride is in the range of 3:1 to 20:1.

22. The method of claim 12, wherein the molar ratio of phase-transfer catalyst to triglyceride is in the range of 0.1 mole % to 5 mole %.

23. The method of claim 12, wherein the molar ratio of base initiator to triglyceride is in the range of 0.1 mole % to 20 mole %.

24. A method for simultaneously synthesizing monoesters and glycerol comprising mixing at least one phase-transfer catalyst, at least one triglyceride, at least one base initiator, and at least one monohydric alcohol.
